(19) United States
(12) Patent Application Publication
(10) Pub. No.: US 2003/0138466 A1
(43) Pub. Date: Jul. 24, 2003

(54) Antioxidant dermatological composition

(75) Inventors: Dileep Bhagwat, Bronxville, NY (US); Bradley P. Glassman, Fairfield, NJ (US); Daniel Glassman, Fairfield, NJ (US)

Correspondence Address: MERCHANT & GOULD PC, P.O. BOX 2903, MINNEAPOLIS, MN, 55402-0903, US

(73) Assignee: Bradley Pharmaceuticals, Inc.

(21) Appl. No.: 09/998,537

(22) Filed: Nov. 28, 2001

(51) Int. Cl. A61K 35/78; A61K 38/05; A61K 31/385; A61K 7/00; A61K 31/355; A61K 31/375; A61K 31/07; A61K 31/17

(52) U.S. Cl. 424/401; 424/766; 514/458; 514/474; 514/18; 514/725; 514/440; 514/588

(57) ABSTRACT

Enhanced dermatological compositions are described herein using from about 21 to about 40 wt-% urea with antioxidant skin protectants suitable for the treatment of icthyosis, xerosis, severely dry skin, dermatitis, eczema, debridement and tissue suffering and other similar skin conditions.

### FIELD OF THE INVENTION

This invention relates to a semi-solid composition containing antioxidants and urea for treating and protecting the skin.

### BACKGROUND OF THE INVENTION

As the outermost layer of skin, the stratum corneum (SC) is continuously exposed to an oxidative environment, including air pollutants, ultraviolet radiation, chemical oxidants, and aerobic microorganisms. Human SC reveals characteristic antioxidant and protein oxidation gradients with increasing antioxidant depletion and protein oxidation towards the outer layers. SC antioxidants, lipids, and proteins are oxidatively modified upon treatments with ultraviolet A/ultraviolet B, ozone, and benzoyl peroxide. Thiele J. J., Schroeter C., Hsieh S. N., Podda M., Packer L., Curr Probl Dermatol. 2001;29:26-42.

Furthermore, the skin is increasingly exposed to ambient UV-irradiation thus increasing its risk for photooxidative damage with long term detrimental effects like photoaging, which is characterized by wrinkles, loss of skin tone, and resilience. Scharffetter-Kochanek K, Brenneisen P, Wenk J, et al., Exp Gerontol. 2000; 35:307-316

The importance of antioxidants is their role in scavenging free radicals, which are created by natural oxidative process occurring in the environment.

There is a need for topical products with antioxidant formulations to prevent UV-induced carcinogenesis and photoaging as well as to modulate desquamatory skin disorders.

### SUMMARY OF THE INVENTION

Accordingly, the present invention includes an improved treatment for the care and protection of the skin, particularly severely dry skin, using a combination of about 21 to about 40 wt-% of urea and about 0.1 to 20 wt-% of an antioxidant in a suitable defined formulation.

Thus, one aspect of the present invention is a dermatological composition including from about 21 to about 40 wt-% urea, from about 0.1 to 20 wt-% of an antioxidant, and the balance being dermatologically acceptable excipients.

The use of such high concentrations of urea combined with antioxidant skin protectants have been found to provide added efficacy and suitability for treating and protecting skin, and, particularly for treating icthyosis, xerosis, severely dry skin, dermatitis, eczema, debridement and tissue softening as well as other skin conditions.

Still another aspect of the present invention is a method of treating xerosis, ichthyosis, severe dry skin, dermatitis, and eczema by applying to skin in need of treatment an effective amount of a semi-solid dermatological composition containing about 21 to about 40 wt-% urea and about 0.1 to 20 wt-% of an antioxidant.

### DETAILED DESCRIPTION

The dermatological composition of the present invention is a semi-solid at room temperature but is easily absorbed into the stratum corneum. A preferred application of the formulation is a cream which contains a petroleum based liquid and solid fraction as skin protectants.

The cream composition has advantageous properties for the treatment of severe dry skin clinically characterized as xerosis and for the temporary relief of itching associated with various pathological dermatological conditions. The formulation produces a keratolytic action found beneficial in the treatment of icthyosis, atopic dermatitis. The formulation also produces added protection against debridement and tissue suffering from oxidative events. Application of the cream to the skin as needed provides relief of the conditions.

In addition to containing about 21 to about 40 wt-% of urea, the composition of the present invention includes an effective amount of antioxidant skin protectants, for example, from about 0.1 to about 20 wt-% based on the total weight of the composition.

Antioxidants include, but are not limited to, tocopherols (vitamin E), tocopherol derivatives, tocotrienols, ascorbic acid (vitamin C), ascorbic acid derivatives, carotenoids, vitamin A or derivatives thereof, butylated hydroxytoluene, butylated hydroxyanisole, gallic esters, flavonoids such as, for example, quercetin or myricetin, selenium, grape seed extract, catechins such as, for example, epicatechin, epicatechingallate, epigallocatechin or epigallocatechingallate, sulfur-containing molecules such as, for example, glutathione, cysteine, lipoic acid, N-acetylcysteine, chelating agents such as, for example, ethylenediamine tetraacetic acid or other customary antioxidants.

One antioxidant, vitamin E, is of particular interest. The term “vitamin E” includes tocopherol (vitamin E) and derivatives thereof such as, for example, α-, β-, γ-, δ-, ε-, ζ₁, ζ₂, and η-tocopherol, and α-tocopherol acid succinate. Vitamin E is known as an antioxidant and protective vitamin for phospholipids of the cell membrane. It maintains the permeability and stability of the cell membrane, Lucy. Annals N. Y Academy of Science 203, p. 4 (1972). It further has been known that vitamin E has a membrane-sealing effect. In erythrocytes, the simplest cells of the human body, there has been found that vitamin E provides a protective effect for the cell membrane. As with all antioxidants, vitamin E protects cells, including, epidermal cells which are susceptible to a wide range of oxidating events.

The cream composition also includes a combination of semi-solid and liquid petroleum fractions. The semi-solid skin protectant is contained in about 5.5 to about 20 wt-% and includes petrolatum or a synthetic or semi-synthetic hydrocarbon of the same nature as petrolatum. Mixtures of such ingredients can also be used. The preferred semi-solid material is petrolatum, commercially available from a wide variety of sources.

The liquid portion skin protectant is a liquid petrolatum and contained in the composition in about 10 to about 20 wt-%. This material can include any synthetic or semi-synthetic oleaginous liquid fraction. A preferred embodiment is mineral oil which is a liquid mixture of hydrocarbons obtained from petroleum.

Another preferred ingredient encompassed in the composition of the present invention is propylene glycol which may be contained up to about 5 wt-% in the composition, preferably in the range of from about 1 to about 5 wt-%.

In addition to the above embodiments, the present composition also contains dermatologically acceptable excipients, such as for example emulsifiers and thickeners. Among these are for example a C₁₆ to C₁₈ straight or branched chain fatty alcohols or fatty acids or mixtures thereof. Preferably these include cetyl alcohol, stearyl alcohol, stearic acid, palmitic acid, or mixtures thereof. Fatty acids or fatty alcohols may be present in from about 0.25 to 2 wt-%.

Another ingredient useful in the composition of the present invention may be glyceryl stearate, which is a monoester of glycerine and stearic acid, or other suitable forms of glyceryl stearate for example glyceryl stearate SE, which is a commercially available self-emulsifying grade of glycerol stearate that contains some sodium and/or potassium stearate. Glyceryl stearate may be in the composition anywhere from about 1 to about 3 wt-%.

Xanthan gum is another ingredient which may be used in the present invention. Xanthan gum is a high molecular weight heteropolysaccharide gum produced by pure-culture fermentation of a carbohydrate with Xanthomonas campestris. The gum is also commercially available from various sources.

As part of the dermatologically acceptable excipients, the composition includes thickeners which provide a high viscosity cream designed to remain in place upon application to the skin. Preferred thickeners include a mixture of a carbomer and triethanolamine. The mixture is combined together and added to the composition in an amount totaling anywhere from about 0.05 to 5 wt-%. Triethanolamine is purchased as Trolamine NF from BASF. The carbomers come in various molecular weights and identified by numbers. These are otherwise known as Carbopol. A preferred embodiment of the present invention is Carbopol 940. The carbomer or Carbopols are resins which are known thickening agents. They are homopolymers of acrylic acid crosslinked with an allyl ether of pentaerythritol, an allyl ether of sucrose or an allyl ether of propylene. The carbomer is present in the composition as a thickener and also is used to suspend and stabilize the emulsion. Although Carbopol 940 is preferably used in the present invention, other analogs may also be used such as carbomer 910, 2984, 5984, 954, 980, 981, 941 and 934. Carbopol ETD 2001, 2020, and 2050 and Ultrez 20 are also commercially available and can be used since they are similar in chemistry and function.

A typical formulation representing the particular and most preferred embodiment of the present invention is illustrated as follows:

| Ingredient | % W/W |
|---|---|
| Anti-oxidant | 5.0 |
| Urea USP | 40 |
| Carbopol 940 | 0.20 |
| Petrolatum | 6.00 |
| Mineral oil | 7.1 |
| Glyceryl stearate | 1.86 |
| Cetyl alcohol | 0.63 |
| Propylene glycol | 3.00 |
| Xanthan gum | 0.05 |
| Trolamine | 0.15 |
| Purified water Q.S. | 100.00. |

### Glossary of Ingredients

The formulation of the present invention has been defined above and more specifically exemplified in the following examples. Since the formulation employs various ingredients, some of the ingredients have been defined generically and by common name. In addition, the following is a glossary of technical names and trade names with manufacturing sources for some of the ingredients employed in the formulation of the present invention.

Mineral Oil

Definition

Mineral oil is a liquid mixture of hydrocarbons obtained from petroleum.

Technical Names

Heavy Mineral Oil

Light Mineral Oil

Liquid Paraffin

Paraffin Oil

Trade Names

Benol White Mineral Oil (Witco/Sonneborn)

Blandol White Mineral Oil (Witco/Sonneborn)

Britol 6 (Witco Corporation)

Britol 7 (Witco Corporation)

Britol 9 (Witco Corporation)

Britol 20 (Witco Corporation)

Britol 24 (Witco Corporation)

Britol 35 (Witco Corporation)

Britol 50 (Witco Corporation)

Carnation White Mineral Oil (Witco/Sonneborn)

Crystosol NF 70 (Witco Corporation)

Crystosol NF 90 (Witco Corporation)

Crystosol U.S. Pat. No. 200 (Witco Corporation)

Crystosol U.S. Pat. No. 240 (Witco Corporation)

Crystosol U.S. Pat. No. 350 (Witco Corporation)

Drakeol 5 (Penreco)

Drakeol 6 (Penreco)

Drakeol 7 (Penreco)

Drakeol 8 (Penreco)

Drakeol 9 (Penreco)

Drakeol 10 (Penreco)

Drakeol 13 (Penreco)

Drakeol 15 (Penreco)

Drakeol 19 (Penreco)

Drakeol 21 (Penreco)

Drakeol 32 (Penreco)

Drakeol 34 (Penreco)

Drakeol 35 (Penreco)

Draketex 50 (Penreco)

Ervol White Mineral Oil (Witco/Sonneborn)

GloriaWhite Mineral Oil (Witco/Sonneborn)

Kaydol White Mineral Oil (Witco/Sonneborn)

Klearol White Mineral Oil (Witco/Sonneborn)

Parol 70 (Penreco)

Parol 80 (Penreco)

Parol 100 (Penreco)

PD-23 White Mineral Oil (Witco/Sonneborn)

Peneteck (Penreco)

Protol White Mineral Oil (Witco/Sonneborn)

Superla Mineral Oil #5 NF (Amoco Lubricants)

Superla Mineral Oil #6 NF (Amoco Lubricants)

Superla Mineral Oil #7 NF (Amoco Lubricants)

Superla Mineral Oil #9 NF (Amoco Lubricants)

Superla Mineral Oil #10 NF (Amoco Lubricants)

Superla Mineral Oil #13 NF (Amoco Lubricants)

Superla Mineral Oil #18 USP (Amoco Lubricants)

Superla Mineral Oil #21 USP (Amoco Lubricants)

Superla Mineral Oil #31 USP (Amoco Lubricants)

Superla Mineral Oil #35 USP (Amoco Lubricants)

Uniwhite Oil 55 (UPI)

Uniwhite Oil 70 (UPI)

Uniwhite Oil 85 (UPI)

Uniwhite Oil 130 (UPI)

Uniwhite Oil 185 (UPI)

Uniwhite Oil 205 (UPI)

Uniwhite Oil 350 (UPI)

Glyceryl Stearate

Empirical Formula

C₂₁H₄₂O₄

Definition

Glyceryl stearate is the monoester of glycerin and stearic acid. It conforms generally to the formula:

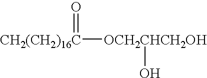

Technical Names

2,3-Dihydroxypropyl octadecanoate

Glyceryl monostearate

Monostearin

Octadecanoic acid, 2,3-dihydroxypropyl ester

Octadecanoic acid, monoester with 1,2,3-propanetriol

Trade Names

Aldo HMS (Lonza Inc./Lonza Ltd.)

Aldo MS (Lonza Inc./Lonza Ltd.)

Aldo MSLG (Lonza Inc./Lonza Ltd.)

Alkamuls GMS (Rhone-Poulenc)

Arlacel 129 (ICI)

Atmos 150 (ICI)

Atmul 84 (ICI)

Atmul 124 (ICI)

Capmul GMS (Karishamns Lipid Specialties)

Ceral MN (Fabriquimica)

Ceral MNT (Fabriquimica)

Cerasynt GMs (ISP Van Dyk)

Cerasynt SD (ISP Van Dyk)

Cithrol GMS N/E (Croda Surfactants Ltd.)

CPH-53-N (Hall)

CPH-144-N (Hall)

Cutina GMS (Henkel)

Cutina MD (Henkel)

Cutina MD-A (Henkel)

Dimodan PM (Grinsted)

Dimodan PM 300 (Grinsted)

Elfacos GMS (Akzo BV)

Emerest 2400 (Henkel/Organic Products)

Empilan GMS NSE (Albright & Wilson)

Emuldan FP 40 (Grinsted)

Emuldan HA 60 (Grinsted)

Emuldan HLT 40 (Grinsted)

ESTOL GMS90 1468 (Unichema)

ESTOL GMSveg 1474 (Unichema)

Geleol (Gattefosse)

Grillomuls S 40 (Grillo-Werke)

Grillomuls S 60 (Grillo-Werke)

Grillomuls S 90 Grillo-Werke)

Hefti GMS-33 (Hefti)

Hefti GMS-99 (Hefti)

Hodak GMS (Calgene)

Imwitor 191 (Huls AG/Huls America)

Imwitor 900 (Huls AG/Huls America)

Kemester 5500 (Witco)

Kemester 6000 (Witco)

Kessco GMS (Akzo BV)

Lanesta 24 (Lanaetex)

Lasemul 92 AE (Industrial Quimica)

Lasemul 92 AE/A (Industrial Quimica)

Lasemul 92 N 40 (Industrial Quimica)

Lexemul 503 (Inolex)

Lexemul 515 (Inolex)

Lexemul 55G (Inolex)

Lipo GMS 410 (Lipo)

Lipo GMS 450 (Lipo)

Lipo GMS 600 (Lipo)

Nikkol MGS-DEX (Nikko)

Norfox GMS (Norman, Fox & Co.)

Norfvox GMS-SE (Norman, Fox & Co.)

Prodhybase GLA (Prod'Hyg)

Protachem 26 (Protameen)

Protachem G 5509 (Protameen)

Protachem G-5566 (Protameen)

Protachem GMS-540 (Protameen)

Protachem HMS (Protameen)

Sterol GMS (Auschem)

Tegin 90 (Goldschmidt)

Tegin 515 (Goldschmidt)

Tegin 4011 (Goldschmidt)

Tegin 4100 (Goldschmidt)

Tegin GRB (Goldschmidt)

Tegin ISO (Goldschmidt)

Tegin M (Goldschmidt)

Tegin MAV (Goldschmidt)

Unitina MD (UPI)

Unitina MD-A (UPI)

Unitolate GS (UPI)

Witconol 2400 (Witco)

Witconol 2401 (Witco)

Witconol MST (Witco SA)

Witconol MST (Witco)

Zohar GLST (Zohar)

Glyceryl Stearate SE

Definition

Glyceryl stearate SE is a self-emulsifying grade of glyceryl stearate (q.v.) that contains some sodium and/or potassium stearate.

Trade Names

Aldo MSD (Lonza Inc./Lonza Ltd.)

Ceral ME (Fabriquimica)

Ceral MET (Fabriquimica)

Ceral TN (Fabriquimica)

Cerasynt Q (ISP Van Dyk)

Cithrol GMS S/E (Croda Surfactants Ltd.)

Cutina KD-16 (Henkel)

Dermalcare GMS/SE (Rhone-Poulenc)

Dracorin GMS SE O/W 2/008475 (Dragoco)

Emerest 2407 (Henkel/Organic Products)

Empilan GMS SE (Albright & Wilson)

Emuldan HA 32/S3 (Grinsted)

ESTOL BMSse 1462 (Unichema)

Hefti GMS-33-SES (Hefti)

Hodag GMS-D (Calgene)

Imwitor 960 (Huls Ag/Huls America)

Kemester 6000 SE (Witco)

Lamecreme KSM (Grunau)

Lanesta 40 (Lanaetex)

Lexemul 530 (Inolex)

Lexemul T (Inolex)

Lipo GMS 470 (Lipo)

Mazol GMSD-K (PPG)

Prodhybase GLN (Prod'Hyg)

REWOMUL MG SE (Rewo Chemische)

Tegin (Goldschmidt)

Tegin Spezial (Goldschmidt)

Tegin V (Goldschmidt)

Unitolate GMS-D (UPI)

Witconol 2407 (Witco)

Cetyl Alcohol

Empirical Formula

C₁₆H₃₄O

Definition

Cetyl alcohol is the fatty alcohol that conforms generally to the formula:

CH₂(CH₂)₁₄CH₂OH

Technical Names

1-Hexadecanol

n-Hexadecyl alcohol

Palmityl alcohol

Trade Names

Adol 52 (Witco)

Adol 520 (Witco)

Adol 52-NF (Witco)

Adol 520-NF (Witco)

Cachalot C-50 (Michel)

Cachalot C-51 (Michel)

Cachalot C-52 (Michel)

Cetaffine (Laserson & Sabetay)

Cetal (Amerchol)

Cetyl alcohol (Rhone-Poulenc)

CO-1695 (Procter & Gamble)

Crodacol C-70 (Croda, Inc.)

Crodacol C90 (Croda Chemicals Ltd.)

Crodacol C-95 (Croda, Inc.)

Fancol CA (Fanning)

Hyfatol 16-95 (Aarhus)

Hyfatol 16-98 (Aarhus)

Lanette 16 (Henkel)

Lanol C (SEPPIC)

Laurex 16 (Albright & Wilson)

Lipocol C (Lipo)

Stearic Acid

Empirical Formula

C₁₈H₃₆O₂

Definition

Stearic acid is the fatty acid that conforms generally to the formula:

CH₂(CH₂)₁₆COOH

Trade Names

Crosterene SA4310 (Croda Universal Ltd.)

Dar-Chem 14 (Darling)

Emersol 120 (Henkel/Emery)

Emersol 132 (Henkel/Emery)

Emersol 150 (Henkel/Emery)

Glycon DP (Lonza Inc./Lonza Ltd.)

Glycon P-45 (Lonza Inc./Lonza Ltd.)

Glycon S-65 (Lonza Inc./Lonza Ltd.)

Glycon S-70 (Lonza Inc./Lonza Ltd.)

Glycon S-90 (Lonza Inc./Lonza Ltd.)

Glycon TP (Lonza Inc./Lonza Ltd.)

Hy-Phi 1199 (Darling)

Hy-Phi 1303 (Darling)

Hy-Phi 1401 (Darling)

Hystrene 4516 (Witco)

Hystrene 5016 (Witco)

Hystrene 7018 (Witco)

Hystrene 9718 (Witco)

Industrene 5016 (Witco)

Industrene 7018 (Witco)

Kartacid 1890 (Akzo BV)

Neo-Fat 18 (Akzo)

Neo-Fat 18-54 (Akzo)

Neo-Fat 18-55 (Akzo)

Neo-Fat 18-61 (Akzo)

Pearl Stearic (Darling)

PRIFAC 2981 (Unichema)

Pristerene 4900 (Unichema)

Pristerene 4901 (Unichema)

Pristerene 4902 (Unichema)

Pristerene 4904 (Unichema)

Pristerene 4905 (Unichema)

Pristerene 4910 (Unichema)

Pristerene 4911 (Unichema)

Pristerene 4915 (Unichema)

Pristerene 4921 (Unichema)

Pristerene 4968 (Unichema)

Pristerene 9550 (Unichema)

Safacid 18 (Pronova)

Safacid 16/18 CR (Pronova)

Unifat 54 (UPI)

Unifat 55L (UPI)

Stearyl Alcohol

Empirical Formula

C₁₈H₃₈O

Definition

Stearyl alcohol is the fatty alcohol that conforms generally to the formula:

CH₃(CH₂)₁₆CH₂OH

Technical Name

1-Octadecanol

Trade Names

Adol 63 (Witco)

Adol 61-NF (Witco)

Adol 62-NF (Witco)

Adol 620-NF (Witco)

Cachalot S-53 (Michel)

Cachalot S-54 (Michel)

Cachalot S-56 (Michel)

CO-1895 (Procter & Gamble)

Crodacol S-70 (Croda, Inc.)

Crodacol S-95 (Croda, Inc.)

Crodacol S-95 (Croda Chemicals Ltd.)

Fancol SA (Fanning)

Hyfatol 18-95 (Aarhus)

Hyfatol 18-98 (Aarhus)

Lanette 18 (Henkel)

Lanol S (SEPPIC)

Laurex 18 (Albright & Wilson)

Lipocol S (Lipo)

Stearal (Amerchol)

Stearyl Alcohol (Rhone-Poulenc)

Steraffine (Laserson & Sabetay)

Unihydag WAX-18 (UPI)

Palmitic Acid

Empirical Formula

C₁₆H₃₂O₂

Definition

Palmitic acid is the fatty acid that conforms generally to the formula:

CH₃(CH₂)₁₄COOH

Technical Name

n-Hexadecanoic acid

Trade Names

Crodacid PD3160 (Croda Universal Ltd.)

Edenor L2SM (Henkel)

Emersol 142 (Henkel/Emery)

Emersol 144 (Henkel/Emery)

Hystrene 7016 (Witco)

Hystrene 9016 (Witco)

Kartacid 1692 (Akzo BV)

Neo-Fat 16 (Akzo)

Neo-Fat 16-54 (Akzo)

Neo-Fat 16-56 (Akzo)

Neo-Fat 16-S (Akzo)

PRIFAC 2962 (Unichema)

Prifrac 2690 (Unichema)

Trade Name Mixture

N.S.L.E. (Sederma)

Propylene Glycol

Empirical Formula

C₃H₈O₂

Definition

Propylene glycol is the aliphatic alcohol that conforms generally to the formula:

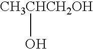

Technical Name

1,2-Propanediol

Trade Names

Lexol PG-865 (855) (Inolex)

1,2-Propylene Glycol USP (BASF)

Xanthan Gum

Definition

Xanthan gum is a high molecular weight hetero polysaccharide gum produced by a pure-culture fermentation of a carbohydrate with Xanthomonas campestris.

Technical Names

Corn sugar gum

Xanthan

Trade Names

Kelgum CG (Calgon)

Keltrol (Kelco)

Keltrol CG (Calgon)

Keltrol CG 1000 (Calgon)

Keltrol CG BT (Calgon)

Keltrol CG F (Calgon)

Keltrol CG GM (Calgon)

Keltrol CG RD (Calgon)

Keltrol CG SF (Calgon)

Keltrol CG T (Calgon)

Keltrol CG TF (Calgon)

Kelzan (Kelco)

Merezan 8 (Meer)

Merezan 20 (Meer)

Rhodigel (Vanderbilt)

Rhodigel (Rhone-Poulenc)

Rhodopol SC (Rhone-Poulenc)

Xanthan gum (Jungbunzlauer)

Triethanolamine

Empirical Formula

C₆H₁₅O₃N

Definition

Triethanolamine is an alkanolamine that conforms generally to the formula:

N(CH₂CH₂OH)₃

Technical Names

Ethanol, 2,2′,2″-Nitrilotris-2,2′,2″-Nitrilotris[Ethanol]

TEA

Trolamine

Trade Name

Triethanolamine Pure C (BASF)

### EXAMPLE

The typical formulation illustrated above is prepared commercially as follows:

| # | Ingredient | Batch | Units |
|---|---|---|---|
| 1 | Purified water | 1084.47 | Gm |
| 2 | Vitamin E | 150.00 | Gm |
| 3 | Urea USP | 1200.00 | Gm |
| 4 | Carbopol 940 | 4.50 | Gm |
| 5 | Petrolatum | 178.20 | Gm |
| 6 | Mineral oil | 211.80 | Gm |
| 7 | Glyceryl stearate | 56.25 | Gm |
| 8 | Cetyl alcohol | 18.78 | Gm |
| 9 | Propylene glycol | 90.00 | Gm |
| 10 | Xanthan gum | 1.50 | Gm |
| 11 | Trolamine NF | 4.50 | Gm |
| 12 | Purified Water Q.S. | 3000.00 | Gm |

The above product was manufactured as follows:

Step 1

Place #1 in Tank A (water phase) and sprinkle in #4 in Tank A and mix to disperse. After uniformly dispersed, heat contents of Tank A to about 75° C. while mixing.

Step 2

Add #3 to Tank A and mix to dissolve.

Step 3

In a separate tank add #2, 5, 6, 7, 8. Heat to about 75° C. with mixing (oil phase).

Step 4

In a separate container disperse uniformly #10 in #9. Add this to Tank A and continue to mix.

Step 5

Add the oil phase (step 3) to the water phase in Tank A with mixing.

Step 6

Add #11 to Tank A and mix. Slowly cool the batch.

Step 7

Add #12 to Q.S. the batch to final weight.

The bulk product is then packaged into conventional containers for use as a cream.

We claim:

1. A dermatological composition comprising:
(a) about 21 to about 40 wt-% urea;
(b) about 0.1 to 20 wt-% of an antioxidant, and the balance being dermatologically acceptable excipients.

2. The composition of claim 1, where the antioxidant selected from the group of tocopherols and derivatives thereof, tocotrenols, ascorbic acid and derivatives thereof, selenium, grape seed extract, carotenoids, vitamin A or derivatives thereof, butylated hydroxytoluene, butylated hydroxyanisole, gallic esters, flavonoids, catechins, glutathione, cysteine, lipoic acid, N-acetylcysteine, ethylenediamine tetraacetic acid and mixtures thereof

3. The composition of claim 1, where the antioxidant is vitamin E.

4. The composition of claim 1, wherein the antioxidant is vitamin C.

5. The composition of claim 2, where the antioxidant is grape seed extract.

6. The composition of claim 1, wherein the excipients comprise skin protectants of an oleaginous nature derived from petroleum, emulsifiers and thickeners.

7. The composition of claim 6, wherein the skin protectants are a mixture of a semi-solid petrolatum or a synthetic or semi-synthetic hydrocarbon or a mixture thereof, and a liquid petrolatum or a synthetic or semi-synthetic oleaginous liquid derivative thereof, or a mixture thereof.

8. The composition of claim 7, wherein the semi-solid petrolatum is present in an amount from about 5.5. to about 20 wt-%.

9. The composition of claim 1, which further comprises up to 5 wt-% of propylene glycol.

10. The composition of claim 1, in the form of a semi-solid at room temperature.

11. The composition of claim 1, which further comprises a mixture of a carbomer and triethanolamine in a total amount from about 0.05 to about 30 wt-%.

12. The composition of claim 1, in the form of a viscous cream, gel or lotion.

13. A method of treating and protecting dry skin comprising applying to skin in need thereof an effective amount of a composition according to claim 1.

14. A method of treating xerosis, icthyosis and severely dry skin comprising applying to skin in need of such treatment an effective amount of a composition according to claim 1.
